(19) United States
(12) Patent Application Publication
(10) Pub. No.: US 2003/0219801 A1
(43) Pub. Date: Nov. 27, 2003

(54) Aptamer base technique for ligand identification

(75) Inventor: Robert Lipshutz, Palo Alto, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/383,957

(22) Filed: Mar. 6, 2003

Related U.S. Application Data
(60) Provisional application No. 60/362,398, filed on Mar. 6, 2002.

(51) Int. Cl. C12Q 1/68; C12P 19/34

(52) U.S. Cl. 435/6; 435/91.2

(57) ABSTRACT

The present invention provides methods to identify target molecules in a biological sample through the use of aptamers designed to bind specific target molecules and by hybridizing nucleic subsequence of the aptamer to a nucleic acid array. The present invention allows the detection and the measure of the amount of the target molecules in a sample and thus can be used for diagnostic medical applications.

### FIELD OF THE INVENTION

The present invention is in the field of molecular biology techniques. More specifically, it is in the field of analyzing protein and target molecule expression.

### BACKGROUND OF THE INVENTION

Many biological functions are accomplished by altering the expression of various genes through transcriptional (e.g. through control of initiation, provision of RNA precursors, RNA processing, etc.) and/or translational control. For example, fundamental biological processes such as cell cycle progression, cell differentiation and cell death, are often characterized by the variations in the expression levels of a group of genes.

Gene expression is also associated with pathogenesis. For example, the lack of sufficient expression of functional tumor suppressor genes and/or the over expression of oncogene/protooncogenes could lead to tumorgenesis (Marshall, Cell, 64: 313-326 (1991); Weinberg, Science, 254: 1138-1146 (1991), incorporated herein by reference for all purposes). Proteins translate genomic sequence information into function, thereby enabling biological processes. Thus, understanding changes in the expression levels of particular genes (e.g. oncogenes or tumor suppressors) and proteins serve as signposts for the presence and progression of various diseases.

Highly parallel methods of monitoring the expression of a large number of genes in a biological sample are a valuable research and diagnostics tool. However, the amount of starting material that can be obtained from a given source is often limited and it is useful to amplify genetic material prior to analysis. Methods of amplification that allow analysis of a sample that may be too small for analysis without amplification facilitate the analysis of gene expression and protein expression in small samples and possibly in a single cell.

### SUMMARY OF THE INVENTION

The present invention relates to the use of an aptamer molecule, designed to bind selectively to a ligand, to capture the specific ligand in a sample, forming an aptamer-ligand complex and subsequent to the separation of the complex from the remainder of the sample and the amplification of a subsequence of the aptamer, to identify the nucleic acid that has captured the ligand through the use of a nucleic acid array and to identify the ligand itself.

Preferably, the nucleic acid has a domain that is recognized by the ligand so that a binding event will occur between the two molecules. It will be known what aptamer will bind to for subsequent analysis. Preferably, the nucleic acid has a sequence that contains a tag that can bind another nucleic acid, preferably on a solid support. The nucleic acid also preferably contains section(s) such as primers that can be used to amplify the tag section. Preferably, the nucleic acid has a sequence or a molecule that will enable it to be reversibly bound to a solid support.

In a preferred embodiment, the aptamer is added to a sample containing a target ligand, such as a protein lysate mixture, under conditions capable of causing the ligand and the aptamer to bind. Then, the aptamer/target is reversibly bound to a solid support, such as a magnetic particle, and separated from the lysate/aptamer mixture. The magnetic particle is removed and the aptamer-ligand complex is purified. Preferably, ligand is labeled specifically and the aptamer-labeled ligand is reversibly bound to a solid support such as a magnetic particle and separated from the aptamer free molecule. The tag sequence is then amplified. The tags are then contacted to a nucleic acid array under hybridization conditions and the array imaged to determine what is bound. By determining what is bound, one can determine which ligands were in the original lysate.

Methods are provided to determine the ligand profile in a biological sample when a collection of aptamers specific to different ligands is used.

In a preferred embodiment, ligand is a polypeptide. In other embodiments, ligand is an hormone, a cofactor, a drug, a toxin or a metabolic byproduct.

In another embodiment, the aptamer sequence contains at least one primer designed to amplify the tag nucleic sequence. Preferably, the tag nucleic sequence is amplified by PCR.

The methods are provided to measure the amount of ligands in a biological sample, wherein the aptamer sequences contain a single set of common primers designed to amplify the tag nucleic sequences that are hybridized a tag probe array and wherein the relative intensity of signal of hybridization is determined.

In an embodiment, the ligands are biotinylated before or after cleavage of the aptamer from the solid support and are purified using an avidin or a streptavidin bound to a solid support such as a magnetic bead.

In another embodiment, the biological sample is a biological fluid, a cell or a tissue lysate and is extracted from virion, bacteria, fungi, algae, plants, animals or humans.

In one embodiment, tag nucleic sequence bear different labels and are hybridized simultaneously to the array.

In another embodiment, the aptamer comprises a moiety that is capable of specifically binding to a moiety on the solid support. Preferably, the moiety system is a biotin-streptavidin system, the solid support is a magnetic bead and the binding between the aptamer-target molecule complex and the solid support is reversible.

In a most preferred embodiment, the ligand profile in different cells or tissues, in different physiological states of the same cells or tissue, at different developmental stages of the same cells or tissue or in different cell populations of the same tissue is determined and compared.

In a preferred embodiment, comparison of hybridization pattern between individuals with and without disease enable the detection of disease such as cancer.

### BRIEF DESCRIPTION OF THE DRAWING

FIG. 1 shows the process of the present invention in a preferred form.

### DETAILED DESCRIPTIONS OF THE INVENTION

I. General

The present invention relies on many patents, applications and other references for details known to those of the art. Therefore, when a patent, application, or other reference is cited or repeated below, it should be understood that it is incorporated by reference in its entirety for all purposes as well as for the proposition that is recited.

As used in the specification and claims, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), all of which are herein incorporated in their entirety by reference for all purposes.

Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, and 6,090,555, which are all incorporated herein by reference in their entirety for all purposes. Nucleic acid arrays are described in many of the above patents, but the same techniques are applied to polypeptide arrays.

Additionally, gene expression monitoring and sample preparation methods can be shown in U.S. Pat. Nos. 5,800,992, 6,040,138, 6,013,449 and 6,309,822. Generally, the present invention contemplates the use of an aptamer molecule, such as a nucleic acid, to capture a target molecule and then identification of the nucleic acid that has captured the target molecule to identify the molecule itself. The target molecule include, but are not limited to, proteins, hormones, sugars, metabolic byproducts, cofactors, drugs and toxins. Preferably, the nucleic acid has a domain that is recognized by the protein so that a binding event will occur between the two molecules. It will be known what aptamer will bind to for subsequent analysis. Preferably, the nucleic acid has a sequence that contains a tag that can bind another nucleic acid, preferably on a solid support. The nucleic acid also preferably contains section(s) that can be used to amplify the tag section. Preferably, the nucleic acid has a sequence or a molecule that will enable it to be reversibly bound to a solid support.

Preferably, the aptamer is added to a sample containing a target polypeptide, such as a protein lysate mixture, under conditions capable of causing the target and the aptamer to bind. Then, the aptamer/target is reversibly bound to a solid support such as a magnetic particle, and separated from the lysate/aptamer mixture. The magnetic particle is removed and then the tag sequence is amplified. The tags are then contacted to a nucleic acid array under hybridization conditions and the array imaged to determined what is bound. By determining what is bound, one can determine which proteins were in the original lysate and can determine a protein expression profile of the original sample.

II. Definitions

Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982)). Indeed, the present invention contemplates any deoxyribonucleotide, ribonucleotide or peptide nucleic acid component, and any chemical variants thereof, such as methylated, hydroxymethylated or glycosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. Oligonucleotide and polynucleotide are included in this definition and relate to two or more nucleic acids in a polynucleotide. (See U.S. Pat. No. 6,156,501 which is hereby incorporated by reference in its entirety.)

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix.

Primer refers to a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

Tag sequence refers a sequence that is attached to reaction product of interest A nucleic acid “tag” is a selected nucleic acid with a specified nucleic acid sequence. Tag-probe refers to the oligonucleotide sequence on the array that is complementary of the Tag sequence. The reaction product on which the Tag has been incorporated will hybridize to the corresponding Tag-probe on the array. Thus, the “tag” nucleic acid functions in a manner analogous to a bar code label, and the nucleic acid array of probes functions in a manner analogous to as a bar code label reader. A set of tags can be, for instance, all possible tags of a specified length (i.e., all 20-mers), or a subset thereof.

Target molecule or Ligand refers to any compound of interest for which an aptamer is desired. A target molecule can be a protein, peptide, carbohydrate, polysaccharide, glycoprotein, hormone, receptor, antigen, antibody, virus, substrate, metabolite, transition state analog, cofactor, inhibitor, drug, dye, nutrient, growth factor, etc., without limitation. Target molecule and ligand terms are used interchangeably in the present invention.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point Tm for the specific sequence at a defined ionic strength and pH. The Tm is the temperature (under defined ionic strength, pH and nucleic acid composition) at which 50% of the probes complementary to the target sequence hybridize to the target sequence at equilibrium. Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2^{nd} Ed. Cold Spring Harbor Press (1989) and Anderson “Nucleic Acid Hybridization” 1^{st} Ed., BIOS Scientific Publishers Limited (1999), which are hereby incorporated by reference in its entirety for all purposes above.

Hybridization probes are oligonucleotides capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Protein or Polypeptides: Protein or Polypeptides are linear arrangements of two or more amino acids. Also included are analogs and derivatives of amino acids that do not affect the functionality of the protein (either “protein” or “polypeptide” will be used interchangeably.)

Aptamers: refer to nucleic acids (typically DNA, RNA or oligonucleotides) that emerge from in vitro selections when the nucleic acid is added to mixtures of molecules. Ligands that bind aptamers are but not limited to proteins, hormones, sugar, metabolic byproducts, cofactors, drugs and toxins. Aptamers of the present invention are preferably specific for a particular molecule and the presence of one may be used to indicate the presence of the other. Aptamers can have diagnostic, target validation and therapeutic applications. The specificity of the binding is defined in terms of the dissociation constant Kd of the aptamer for its ligand. Aptamers can have high affinity with Kd range similar to antibody (pM to nM) and specificity similar/superior to antibody (Tuerk and Gold, Science, 249: 505 (1990); Ellington and Szostak, Nature, 346:818 (1990)). An aptamer will typically be between 10 and 300 nucleotides in length. Their small size compared to antibodies make them more amenable to dense packing in arrays. RNAs and DNAs aptamers can be generated from in vitro selection experiments such as SELEX (Systematic Evolution of Ligands by Exponential Enrichment). Examples of aptomer uses and technology are PhotoSELEX™ and Riboreporters™.

Aptamers, their uses and manufacture are shown in U.S. Pat. Nos. 5,840,867, 6,001,648, 6225,058, 6,207,388 and U.S. Patent publication 20020001810 all of which are incorporated by reference in their entireties.

Array: An array comprises a support, preferably solid, with nucleic acid probes attached to said support. Arrays typically comprise a plurality of different nucleic acid probes that are coupled to a surface of a substrate in different, known locations. These arrays, also described as “microarrays” or colloquially “chips” have been generally described in the art, for example, U.S. Ser. No. 09/536,841, WO 00/58516, U.S. Pat. Nos. 5,143,854, 5,445,934, 5,744,305, 5,677,195, 6,040,193, 5,424,186 and Fodor et al., Science, 251:767-777 (1991), each of which is incorporated by reference in its entirety for all purposes. These arrays may generally be produced using mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, 6,121,048, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.)

Arrays may be packaged in such a manner as to allow for diagnostics or can be an all-inclusive device; e.g., U.S. Pat. Nos. 5,856,174 and 5,922,591 incorporated in their entirety by reference for all purposes. (See also U.S. patent application Ser. No. 09/545,207 for additional information concerning arrays, their manufacture, and their characteristics.) It is hereby incorporated by reference in its entirety for all purposes.

Preferred arrays are commercially available from Affymetrix under the brand name GeneChip® and are directed to a variety of purposes, including Tag arrays (GenFlex®), gene expression monitoring for a variety of eukaryotic and prokaryotic species. (See Affymetrix Inc., Santa Clara, Calif. and their website at affymetrix.com.)

III. Methods

The current identification and characterization of molecules such as proteins uses a variety of technologies which are both costly and time-consuming, such as the use of monoclonal antibodies, electrophoresis, 2-dimensional electrophoresis analysis, mass spectrographic analysis and protein sequencing. Proteomics studies will benefit from the development of protein arrays and antibody arrays. However, although automation started to be introduced to the processing of 2-dimensional electrophoresis, problem regarding the production and stability of antibodies and the array is still an obstacle for high throughput protein analysis. It is therefore desirable to provide a method that overcomes the before-mentioned inadequacies of the above techniques.

The present invention provides a rapid method for identifying and characterizing molecules such as proteins and proteins profile of a large number of molecules without the need of cloning, over expressing or isolating the molecules of interest.

The method relies on aptamers which are ligand-binding nucleic acids that can recognize selectively and with high affinity target molecules. The development in combinatorial chemistry have resulted in the creation of library of aptamers (For recent reviews on aptamers and their ligands, see Eaton, Curr. Opin. Chem. Biol., 1: 10-16 (1997) and Herrmann and Patel, Science, 287: 820-825 (2000). Selection is based on the ability to bind target molecule such as polypeptide with high affinity and selectivity. Photo-cross-linkable aptamers can be cross-linked to their cognate targets upon brief exposure to ultraviolet (UV) light. Consequently, aptamers have the potential role to fulfill the role that antibodies play with additional advantages. Aptamers are identified through in vitro process and the properties of the aptamers can be tailored on demand. Selection conditions such as incubation buffers and temperature can be manipulated to obtain aptamers with properties that will suit the in vitro reaction. Furthermore, molecules that do not elicit good immune response such as toxins can bind aptamers.

The present invention is also capable of distinguishing ligands in different conformations since aptamers can be raised to discriminate between closely related substances (i.e small structural changes such as a methyl group, a hydroxyl group and a urea versus a guanidine group). Also, aptamers can distinguish between different binding sites of a same ligand.

In the present invention, the molecule of interest is screened as an aptamer-target molecule complex and is identified by a portion of its nucleic acid called tag sequence. Alternatively, multiple aptamers may be used simultaneously against multiple proteins. Tag nucleic acid sequences can be attached to the aptamers at specific location.

In the present invention, the aptamers contains a Tag nucleic sequence. Desirable nucleic acid tag sets have several properties (See U.S. Pat. No. 6,458,530 which is hereby incorporated by reference in its entirety). Preferably, the tag sets have uniform hybridization characteristics (i.e., similar thermal binding stability to complementary nucleic acids), making the tag sets suitable for detection by nucleic acid probe arrays, such as the GenFlex® or GeneChip® nucleic acid probe array. Because the hybridization characteristics of the tags are uniform, all of the tags in the set are typically detectable using a single set of hybridization and wash conditions. Individual tags hybridize only to their complementary probes, and do not significantly cross hybridize with probes complementary to other sequence tags. Most typically, tags are between 8 and 100 nucleotides in length, and preferably between about 10 and 30 nucleotides in length. Most preferably, the tags are between 15 and 25 nucleic acids in length. For example, in one preferred embodiment, the nucleic acid tags are about 20 nucleotides in length.

The tags are selected so that no tag hybridizes to a probe with only one sequence mismatch (all tags differ by at least two nucleotides). Optionally, tags can be selected which have at least 2 mismatches, 3 mismatches, 4 mismatches 5 mismatches or more to a probe which is not perfectly complementary to the tag, depending on the application. Typically, all tag sequences are selected to hybridize only to a perfectly complementary probe, and the nearest mismatch hybridization possibility has at least two hybridization mismatches. Thus, the tag sequences typically differ by at least two nucleotides when aligned for maximal correspondence. Preferably, the tags differ by about 5 nucleotides when aligned for maximal correspondence (e.g., where the tags are 20-mers).

The tags are often selected so that they do not have identical runs of nucleotides of a specified length. For instance, where the tags are 20-mers, the tags are preferably selected so that no two tags have runs of about 9 or more nucleotides in common. One of skill will appreciate that the length of prohibited identity varies depending on the selected length of the tag. It was empirically determined that cross-hybridization occurs in tag sets when 20-mer tags have more than about 8 contiguous nucleotides in common.

The tags are selected so that there is no secondary structure within the complementary probes used to detect the tags which are complementary to the tags. Typically, this is done by eliminating tags from a selected tag set which have subsequences of 4 or more nucleotides which are complementary. The tags are selected so that no secondary structure forms between a tag and any associated aptamer nucleic sequence. Self-complementary tags have poor hybridization properties in arrays, because the complementary portions of the probes (and corresponding tags) self hybridize (i.e., form hairpin structures).

The tags are selected so that probes complementary to the tags do not hybridize to each other, thereby preventing duplex formation of the tags in solution.

The methods of the invention use a moiety that is capable of specifically binding to the aptamer to a solid support. See U.S. Pat. No. 6,348,318 which is hereby incorporated by reference in its entirely. A moiety can be, for example, a biotin/streptavidin system which is known to one of skill in the art. It can also be other moieties which can reversibly bind the aptamer to a solid support such as a polypeptide, such as an antibody, or an antibody fragment, that recognizes the aptamer. Various procedures known in the art can be used for the production of antibodies that specifically bind to a particular target analyte (See the references cited in U.S. Pat. No. 6,348,318). Nucleic acids or other chemical entities may be used to bind the complex to a support for subsequent separation.

The solid support above is used to separate the aptamer/target molecule complex from the original mixture. So, the solid support should be designed to facilitate this separation. Preferably, this solid support can be a magnetic bead. Magnetic beads or particles, such as magnetic latex beads and iron oxide particles, that are useful in the claimed invention are known to those of skill in the art. For example, magnetic particles are described in U.S. Pat. No. 4,672,040. Magnetic particles are commercially available from, for example, PerSeptive Biosystems, Inc. (Framingham Mass.), Ciba Coming (Medfield Mass.), Bangs Laboratories (Carmel Ind.), and BioQuest, Inc. (Atkinson N.H.). Coupling of capture moieties to magnetic beads can be accomplished using known methods. For example, beads are commercially available that are derivatized with amino or carboxyl groups that are available for linkage to a protein or other capture moiety using, for example, glutaraldehyde, carbodiimide, diazoto compounds, or other suitable crosslinking reagent. Silanization of magnetically responsive particles provides one method of obtaining reactive groups on the surface of the particles (see, e.g., U.S. Pat. No. 4,672,040 for a description of silanization and silane coupling chemistry). Linking bonds can include, for example, amide, ester, ether, sulfonalmide, disulfide, azo, and others known to those of skill in the art. In one embodiment, the magnetic beads are iron oxide particles that are silanized. An example of suitable silanized beads having functional groups appropriate for covalent linking of capture moieties is the BioMag.™. particle that is commercially available from PerSeptive Biosystems, Inc. Although covalent linkage of the anchor moiety to the magnetic bead is generally preferred, noncovalent linkages are also useful in the claimed methods and kits. For example, capture moieties can be attached to magnetic latex beads through non-covalent physical adsorption.

The capture moiety is capable of specifically binding, in a reversible manner, to the moiety on the aptamer. Several systems are mentioned in U.S. Pat. No. 6,348,318 which is hereby incorporated by reference in its entirely.

Once the aptamer is bound to the solid support such as a magnetic particle, it is separated from the mixture using conventional technology for separating these particles. Then the aptamer is released from the bead. The binding moiety can be engineered to release or cleavage sites can be incorporated into the aptamer. Cleavage sites may be engineered in the aptamer as unique sequences that are recognized by enzymes such as restriction enzymes. Many other commercially available systems can be used.

The methods of the invention use a label capable of specifically binding the ligand to separate the aptamer-ligand complexes from the free aptamers. In one embodiment the aptamer-ligand complex is labeled before the aptamer is released from the solid support. The methods of the invention uses a label that is capable to bind specifically proteins. Proteins labeling techniques are well known in the art. Biotin, for example, is being widely used in protein chemistry and enables detection, purification and/or immobilization of the protein using the avidin or streptavidin system. Biotin and molecule derived from biotin react with primary amines of proteins and other molecules to form stable biotin conjugates. Following biotinylation, unreacted biotin may be removed by washing the solid phase, and its retained protein. Aptamer-protein complexes can then be cleaved from the solid support as discussed above. Alternatively the aptamer-protein complex is cleaved from the solid support before biotinylation. The high affinity and specificity of avidin-biotin or streptavidin-biotin interactions enables the purification of the aptamer-biotinylated complex using avidin or streptavidin coated solid support system which is known to one of skill in the art.

The solid support above is used to separate the aptamer/target molecule complex from the free aptamer. So, the solid support should be designed to facilitate this separation. Preferably, this solid support can be a magnetic bead as described above.

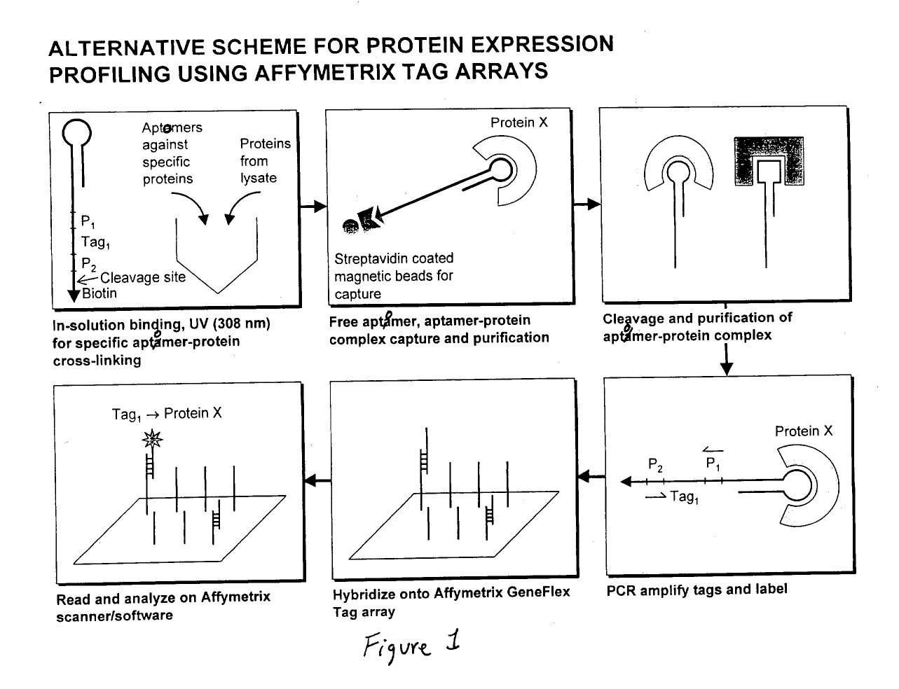

Another advantage of the present invention is the detection and identification of small amount of ligands since the nuclei acid based part of the nucleic acid-ligand complex is amplified. Preferably, the aptamer contains one or more primers to amplify a tag sequence that is present on the aptamer. For instance, a nucleic acid tag is in proximity to PCR primer binding sites which, when amplified using standard PCR techniques, amplifies the tag nucleic acid, or a subsequence thereof. Preferably there are two primer sequences adjacent or near the tag sequence, one for each direction as shown in FIG. 1. Thus, tagged aptamers can be detected even if the tag nucleic acids are present in very small quantities. One of skill will appreciate that a single molecule of a nucleic acid tag can easily be detected after amplification.

Preferably, the tag nucleic sequence is amplified using a single set of common primers and can be quantitatively identified by hybridization (see, e.g., Shoemaker et al., 1996 Nature Genetics 14:450). The relative intensity of the hybridization signals for each tag is then determined according to methods well known in the art.

There are many known methods of amplifying nucleic acid sequences including e.g., PCR. See, e.g., PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. Nos. 4,683,202, 4,683,195, 4,800,159 4,965,188 and 5,333,675 each of which is incorporated herein by reference in their entireties for all purposes.

PCR is an extremely powerful technique for amplifying specific polynucleotide sequences, including genomic DNA, single-stranded cDNA, and mRNA among others. Various methods of conducting PCR amplification and primer design and construction for PCR amplification will be known to those of skill in the art. Generally, in PCR a double stranded DNA to be amplified is denatured by heating the sample. New DNA synthesis is then primed by hybridizing primers to the target sequence in the presence of DNA polymerase and excess dNTPs. In subsequent cycles, the primers hybridize to the newly synthesized DNA to produce discreet products with the primer sequences at either end. The products accumulate exponentially with each successive round of amplification.

The DNA polymerase used in PCR is often a thermostable polymerase. This allows the enzyme to continue functioning after repeated cycles of heating necessary to denature the double stranded DNA. Polymerases that are useful for PCR include, for example, Taq DNA polymerase, Tth DNA polymerase, Tfl DNA polymerase, Tma DNA polymerase, Tli DNA polymerase, and Pfu DNA polymerase. There are many commercially available modified forms of these enzymes including: AmpliTaq® and AmpliTaq Gold® both available from Applied Biosystems. Many are available with or without a 3- to 5′ proofreading exonuclease activity. See, for example, Vent® and Vent® (exo-) available from New England Biolabs.

Other suitable amplification methods include the ligase chain reaction (LCR) (e.g., Wu and Wallace, Genomics 4, 560 (1989) and Landegren et al., Science 241, 1077 (1988)), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NABSA). (See, U.S. Pat. Nos. 5,409,818, 5,554517, and 6,063,603). The latter two amplification methods include isothermal reactions based on isothermal transcription, which produce both single-stranded RNA (ssRNA) and double-stranded DNA (dsDNA) as the amplification products in a ratio of about 30 or 100 to 1, respectively.

The reaction vessel according to the present invention may include a membrane, filter, microscope slide, microwell, sample tube, array, or the like. (See International Patent applications No. PCT/US95/07377 and PCT/US96/11147, which are expressly incorporated herein by reference.) The reaction vessel may be made of various materials, including polystyrene, polycarbonate, plastics, glass, ceramic, stainless steel, or the like. The reaction vessel may preferably have a rigid or semi-rigid surface, and may preferably be conical (e.g., sample tube) or substantially planar (e.g., flat surface) with appropriate wells, raised regions, etched trenches, or the like. The reaction vessel may also include a gel or matrix in which polypeptides may be embedded. (See A. Mirzabekov et al., Anal. Biochem. 259(1):34-41 (1998), which is expressly incorporated herein by reference.)

In a preferred embodiment, the target molecules of the present invention may be analyzed with a gene expression monitoring system. Several such systems are known. A preferred gene expression monitoring system according to the present invention may be a polypeptide or nucleic acid probe array, such as the GenFlex® or GeneChip® nucleic acid probe array (Affymetrix, Santa Clara, Calif.). (See, U.S. Ser. No. 09/536,841, WO 00/58516, U.S. Pat. Nos. 5,744,305, 5,445,934, 5,800,992, 6,040,193 and International Patent applications PCT/US95/07377, PCT/US96/14839, and PCT/US96/14839, which are expressly incorporated herein by reference. A nucleic acid probe array preferably comprises nucleic acids bound to a substrate in known locations. In other embodiments, the system may include a solid support or substrate, such as a membrane, filter, microscope slide, microwell, sample tube, bead, bead array, or the like. The solid support may be made of various materials, including paper, cellulose, gel, nylon, polystyrene, polycarbonate, plastics, glass, ceramic, stainless steel, or the like including any other support cited in U.S. Pat. Nos. 5,744,305 or 6,040,193. The solid support may preferably have a rigid or semi-rigid surface, and may preferably be spherical (e.g., bead) or substantially planar (e.g., flat surface) with appropriate wells, raised regions, etched trenches, or the like. The solid support may also include a gel or matrix in which nucleic acids may be embedded. The gene expression monitoring system, in a preferred embodiment, may comprise a nucleic acid probe array (including an oligonucleotide array, a cDNA array, a spotted array, and the like), membrane blot (such as used in hybridization analysis such as Northern, Southern, dot, and the like), or microwells, sample tubes, beads or fibers (or any solid support comprising bound nucleic acids). See U.S. Ser. No. 09/536,841, WO 00/58516, U.S. Pat. Nos. 5,770,722, 5,744,305, 5,677,195 5,445,934, and 6,040,193 which are incorporated here in their entirety by reference. The gene expression monitoring system may also comprise nucleic acid probes in solution.

Once the tag has been amplified, it is contacted with an array under hybridization conditions. Both strands of a double-stranded tag amplification product are separately monitored by the probe array. Hybridization of each of the strands of the double-stranded tag provides an independent readout for the presence or absence of the tag nucleic acid in a sample. See the patents and applications referred to above. Specifically, see U.S. Ser. No. 09/536,841, WO 00/58516 and U.S. Pat. Nos. 5,143,854 for an example array and 6,309,822 and the publications by Lockhart et al. above for hybridization conditions.

The array is imaged using conventional techniques as shown in the patents referred to above. Imaging equipment is commercially available from Affymetrix Inc. Once the array is imaged, it will be known which aptamer had a bound ligand. Since one would know what aptamer binds to what specific ligand, then the particular ligands of the mixture will be identified and then a ligand profile can be generated. Therefore, identifying the nucleic tag sequence provides an identification of the ligand that bound the aptamer compound. In a preferred embodiment, a protein expression profile is generated.

After the molecule profile such as protein expression profile has been determined the data can be analyzed using any of a number of techniques such as those shown in U.S. Pat. Nos. 6,308,170 and 6,229,911 which are hereby incorporated by reference in their entireties.

Differential ligands and protein profiles have been used as molecular diagnostics for diseases such as cancer and autoimmune diseases and might ultimately be applied to screening of high-risk and general populations. The ligand monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue. (See U.S. Pat. Nos. 5,800,922, 6,228,575, and 6,040,138 which are hereby incorporated by reference in their entireties.)

The ligand population such as polypeptide population of the present invention may be obtained or derived from any tissue or cell source. Indeed, the ligand population may be obtained from any biological or environmental source, including plant, virion, bacteria, fungi, or algae, from any sample, including body fluid or soil. In one embodiment, eukaryotic tissue is preferred, and in another, mammalian tissue is preferred, and in yet another, human tissue is preferred. The tissue or cell source may include a tissue biopsy sample, a cell sorted population, cell culture, or a single cell. In a preferred embodiment, the tissue source may include brain, liver, heart, kidney, lung, spleen, retina, bone, lymph node, endocrine gland, reproductive organ, blood, nerve, vascular tissue, and olfactory epithelium. In yet another preferred embodiment, the tissue or cell source may be embryonic or tumorigenic.

Tumorigenic tissue according to the present invention may include tissue associated with malignant and pre-neoplastic conditions, not limited to the following: acute lymphocytic leukemia, acute myelocytic leukemia, myeloblastic leukemia, promyelocytic leukemia, myelomonocytic leukemia, monocytic leukemia, erythroleukemia, chronic myelocytic (granulocytic) leukemia, chronic lymphocytic leukemia, polycythemia vera, lymphoma, Hodgkin's disease, non-Hodgkin's disease, multiple myeloma, Waldenstrom's macroglobulinemia, heavy chain disease, solid tumors, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. (See Fishman et al., Medicine, 2d Ed. (J. B. Lippincott Co., Philadelphia, Pa. 1985), which is expressly incorporated herein by reference.)

Aptamers have been evolved to bind proteins which are associated with a number of disease states. Using the method of the present invention, many powerful antagonists of such proteins can be found.

Those skilled in the art will recognize that the products and methods embodied in the present invention may be applied to a variety of systems, including commercially available gene expression monitoring systems involving nucleic acid probe arrays, membrane blots, microwells, beads, and sample tubes, constructed with various materials using various methods known in the art. Accordingly, the present invention is not limited to any particular environment, and the following description of specific embodiments of the present invention are for illustrative purposes only.

What is claimed is:

1. A method for detecting the presence of a ligand in a biological sample which may contain said ligand, the method comprising:
a. exposing said biological sample to a capture aptamer capable of binding to said ligand, wherein said capture aptamer contains a tag nucleic sequence that is complementary to a tag-probe and a moiety that can bind on a solid support;
b. generating an aptamer-ligand complex;
c. binding said aptamer-ligand complex on the solid support and separating said complex from the remainder of the biological sample;
d. separating aptamer-ligand complex from free aptamer and separating aptamer-ligand complex from solid support;
e. amplifying the tag nucleotide sequence;
f. providing a nucleic acid array comprising tag probes designed to interrogate said tag nucleic sequences;
g. hybridizing amplified tag sequences to the array;
h. analyzing the hybridization pattern to identify at least one aptamer; and
i. identifying at least one ligand corresponding to said identified aptamer.

2. A method of determining a ligand profile of a biological sample, the method comprising:
a. providing a collection of aptamers specific to ligands to be identified, wherein said aptamers contain different tag nucleic sequences capable of hybridizing to a tag probe and a moiety capable of binding on a solid support;
b. providing an array of tag probes that are complementary to the tag sequence of the apatmers;
c. exposing said biological sample to the collection of aptamers;
d. generating aptamer-ligand complexes;
e. binding said aptamer-ligand complexes on the solid support and separating said complex from the remainder of the biological sample;
f. separating aptamer-ligand complex from free aptamer and separating aptamer-ligand complex from solid support;
g. amplifying the tag nucleic sequences;
h. hybridizing amplified tag sequences to said array;
i. analyzing the hybridization pattern to identify a plurality of aptamers;
j. identifying a plurality of ligands corresponding to said identified aptamers.

3. A method for detecting a disease, the method comprising;
a. identifying aptamers specific to at least one ligand that may be indicative of a disease, wherein said aptamers contain different tag nucleic sequences capable of hybridizing to a tag probe and a molecule capable of binding a solid support;
b. binding aptamers to biological samples that may comprise said ligand from individuals with or without said disease;
c. generating aptamer-target molecule complexes;
d. binding said aptamer-ligand complexes on the solid support and separating said complex from the remainder of the biological sample;
e. separating aptamer-ligand complex from free aptamer and separating aptamer-ligand complex from solid support;
f. providing an array of tag probes that are complementary to the tag nucleic sequence of the apatmers;
g. amplifying the tag nucleotide sequences;
h. hybridizing amplified tag sequences to the array;
i. generating an hybridization pattern for individuals with the disease and an hybridization pattern for individuals without the disease;
j. comparing the hybridization patterns;
k. identifying the ligand(s) corresponding to the aptamer(s) that are specific a disease.

4. A method according to claim 3 wherein the disease is cancer.

5. A method according to claim 1 wherein the capture aptamer is an RNA, DNA, or an oligonucleotide.

6. A method according to claim 2 wherein the capture aptamer is an RNA, DNA, or an oligonucleotide.

7. A method according to claim 3 wherein the capture aptamer is an RNA, DNA, or an oligonucleotide.

8. A method according to claim 1 wherein the ligand is a polypeptide, hormone, cofactor, drug, toxin or a metabolic byproduct.

9. A method according to claim 2 wherein the ligand is a polypeptide, hormone, cofactor, drug, toxin or a metabolic byproduct.

10. A method according to claim 1 wherein the aptamer sequence contains at least one primer designed to amplify the tag nucleic sequence.

11. A method according to claim 2 wherein the aptamer sequence contains at least one primer designed to amplify the tag nucleic sequence.

12. A method according to claim 3 wherein the aptamer sequence contains at least one primer designed to amplify the tag nucleic sequence.

13. A method according to claim 1 further measuring the amount of ligands in a biological sample, wherein
a. the aptamer sequences contain a single set of common primers designed to amplify the tag nucleic sequences;
b. contacting a tag probe array with the amplified tag nucleic sequences; and
c. determining the relative intensity of signal of hybridization of the amplified tag sequence to said array.

14. A method according to claim 12 wherein the tag nucleic sequence is amplified by PCR.

15. A method according to claim 13 wherein the tag nucleic sequence is amplified by PCR.

16. A method according to claim 1 wherein the aptamer comprises a moiety that is capable of specifically binding to a moiety on the solid support.

17. A method according to claim 2 wherein the aptamer comprises a moiety that is capable of specifically binding to a moiety on the solid support.

18. A method according to claim 3 wherein the aptamer comprises a moiety that is capable of specifically binding to a moiety on the solid support.

19. A method according to claim 16 wherein the moiety system is a biotin-streptavidin system.

20. A method according to claim 17 wherein the moiety system is a biotin-streptavidin system

21. A method according to claim 19 wherein the solid support is a magnetic bead.

22. A method according to claim 1 wherein the biological sample is a biological fluid, cell or a tissue lysate.

23. A method according to claim 1 wherein the biological sample is extracted from virion, bacteria, fungi, algae, plants, animals or humans.

24. A method according to claim 2 wherein the biological sample is extracted from virion, bacteria, fungi, algae, plants, animals or humans.

25. A method according to claim 3 wherein the biological sample is extracted from virion, bacteria, fungi, algae, plants, animals or humans.

26. A method according to claim 1 wherein tag nucleic sequences bear different labels and are hybridized simultaneously to the array.

27. A method according to claim 2 further comparing the ligand profile in different cells or tissues.

28. A method according to claim 2 further comparing the ligand profile in different physiological states of the same cells or tissue.

29. A method according to claim 2 further comparing the ligand profile at different developmental stages of the same cells or tissue.

30. A method according to claim 2 further comparing the ligand profile in different cell populations of the same tissue.

31. A method of detecting a ligand in a sample, comprising
a. providing an aptamer that is designed to bind to a ligand;
b. contacting the aptamer with a biological sample that contains the ligand, under conditions that generate a aptamer/ligand complex;
c. separating the complex from the remainder of the sample;
d. amplifying a subsequence of the aptamer;
e. contacting the subsequence with a nucleic acid array under hybridization conditions; and
f. identifying the ligand.

32. A method of detecting at least one target molecule in a sample, comprising
a. providing a nucleic acid that is designed to bind at least one target molecule;
b. contacting the nucleic acid with a biological sample that contains the target molecule(s);
c. separating the complex from the remainder of the sample;
d. amplifying a subsequence of the nucleic acids;
e. identifying at least one target molecule through the use of an array.

33. A method of detecting at least one target molecule in a sample, comprising:
a. providing a nucleic acid that is designed to bind at least one target molecule;
b. contacting the nucleic acid with a biological sample that contains the target molecule(s);
c. separating the complex from the remainder of the sample;
d. detecting at least one nucleic acid through the use of an array; and
e. identifying at least one ligand.

34. A method for detecting a target molecule in a biological sample, the method comprising:
a. generating a nucleic acid array, designed to interrogate subsequence of at least one aptamer;
b. contacting the nucleic acid with a biological sample that contains the target molecule(s) and generating at least one aptamer-target molecule complex;
c. separating the complexes from the remainder of the sample;
d. amplifying a subsequence of said aptamer-target molecule complexes;
e. contacting the array with said amplified aptamer subsequences; and
f. identifying at least one target molecule.
g. ach nucleic acid being hybridized to an aptamer-target molecule complex, wherein the aptamers are designed to bind at least one target molecule present in a biological sample;
h. detecting and measuring the amount of said target molecule.

35. A method according to claim 31 wherein the capture aptamer is an RNA, DNA, or an oligonucleotide.

36. A method according to claim 31 wherein the aptamer sequence contains at least one primer designed to amplify the tag nucleic sequence.

37. A method according to claim 33 wherein the aptamer comprises a moiety that is capable of specifically binding to a moiety on the solid support.

38. A method according to claim 37 wherein the moiety system is a biotin-streptavidin system.

39. A method according to claim 31 wherein the biological sample is extracted from virion, bacteria, fungi, algae, plants, animals or humans.

40. A method according to claim 31 further comparing the ligand profile in different cells or tissues.
